(19) United States
(12) Patent Application Publication
(10) Pub. No.: US 2023/0259864 A1
(43) Pub. Date: Aug. 17, 2023

(54) WORKPLACE ENHANCEMENT VIA DIGITAL TWIN-BASED SIMULATION

(71) Applicant: International Business Machines Corporation, Armonk, NY (US)

(72) Inventors: Clement Decrop, Arlington, VA (US); Jeremy R. Fox, Georgetown, TX (US); Tushar Agrawal, West Fargo, ND (US); Sarbajit K. Rakshit, Kolkata (IN)

(21) Appl. No.: 17/670,656

(22) Filed: Feb. 14, 2022

(51) Int. Cl. G06Q 10/06; A61B 5/00; A61B 5/01; A61B 5/08

(52) U.S. Cl. CPC G06Q 10/067; G06Q 10/06395; A61B 5/0022; A61B 5/01; A61B 5/0823; A61B 2503/20; A61B 2560/0242

(57) ABSTRACT

An approach for enhancing a workplace environment is provided. Environmental information about the workplace environment is obtained from a plurality of internet of things (IoT) device sensors. Based on the environmental information, a digital twin of the workplace environment is created. The digital twin is evaluated over time using a cognitive system to identify a deficiency in the workplace environment using continuous learned data. Based on the evaluation, an area of an avatar-based version of the digital twin that contains a graphical change in the digital twin containing a suggested improvement that addresses the deficiency in the workplace environment is highlighted.

The present invention relates to enhancement of a workplace. Specifically, the present invention relates to utilizing digital twin-based simulation to enhance the ergonomics and other user-experience aspects of a workplace to improve a user experience.

### BACKGROUND

In various working environments, different types of workers are often needed to perform distinct types of activities, with some of the activities being manual, others being automatic, and still others being semi-automatic. Because of this, there is no ideal workplace design that is optimal for every workplace. It is generally understood that providing a working environment with a pleasant working experience and superior ergonomics can ensure better worker satisfaction and productivity. In addition, many health concerns are work related, being caused or accentuated by features of the workplace environment. Many of these healthcare concerns may not be immediately evident and may only present themselves over the long term. For these reasons, deficiencies in the workplace experience may lead to lost productivity and/or worker discontent, among other things.

The Internet of Things (IoT) describes a network of physical objects (things”) that are embedded with sensors, software, and other technologies for the purpose of connecting and exchanging data with other devices and systems over the Internet. By combining these connected devices with automated systems, it is possible to gather information, analyze it and create an action to help someone with a particular task, or learn from a process.

As generally understood, in computing systems, a digital twin is a virtual replica of a physical product, process, or system that can help bridge physical and digital worlds. In essence, a digital twin is a computer program that takes real-world data about a physical object or system as inputs and produces as outputs predications or simulations of how that physical object or system will be affected by those inputs. This allows the digital twin to simulate the physical object in real time, in the process offering insights into performance and potential problems.

### SUMMARY

Embodiments of the invention present invention provide an approach for enhancing a workplace environment. Environmental information about the workplace environment is obtained from a plurality of internet of things (IoT) device sensors. Based on the environmental information, a digital twin of the workplace environment is created. The digital twin is evaluated over time using a cognitive system to identify a deficiency in the workplace environment using continuous learned data. Based on the evaluation, an area of an avatar-based version of the digital twin that contains a graphical change in the digital twin containing a suggested improvement that addresses the deficiency in the workplace environment is highlighted.

One aspect of the present invention includes a computer-implemented method for enhancing a workplace environment, comprising the computer-implemented steps of: obtaining environmental information about the workplace environment from a plurality of internet of things (IoT) device sensors; creating a digital twin of the workplace environment based on the environmental information; evaluating the digital twin over time using a cognitive system to identify a deficiency in the workplace environment using continuous learned data; and highlighting an area of an avatar-based version of the digital twin that contains a graphical change in the digital twin containing a suggested improvement that addresses the deficiency in the workplace environment.

A second aspect of the present invention provides a system for enhancing a workplace environment, comprising: a memory medium comprising program instructions; a bus coupled to the memory medium; and a processor, for executing the program instructions, coupled to the memory medium that when executing the program instructions causes the system to: obtain environmental information about the workplace environment from a plurality of internet of things (IoT) device sensors; create a digital twin of the workplace environment based on the environmental information; evaluate the digital twin over time using a cognitive system to identify a deficiency in the workplace environment using continuous learned data; and highlight an area of an avatar-based version of the digital twin that contains a graphical change in the digital twin containing a suggested improvement that addresses the deficiency in the workplace environment.

A third aspect of the present invention provides a computer program product for enhancing a workplace environment, the computer program product comprising: a computer readable storage device, and program instructions stored on the computer readable storage media, to: obtain environmental information about the workplace environment from a plurality of internet of things (IoT) device sensors; create a digital twin of the workplace environment based on the environmental information; evaluate the digital twin over time using a cognitive system to identify a deficiency in the workplace environment using continuous learned data; and highlight an area of an avatar-based version of the digital twin that contains a graphical change in the digital twin containing a suggested improvement that addresses the deficiency in the workplace environment.

### BRIEF DESCRIPTION OF THE DRAWINGS

These and other features of this invention will be more readily understood from the following detailed description of the various aspects of the invention taken in conjunction with the accompanying drawings in which:

FIG. 1 shows an architecture in which the invention may be implemented according to an embodiment of the present invention;

FIG. 2 shows a system diagram describing the functionality discussed herein according to an embodiment of the present invention;

FIG. 3 shows an example workplace environment according to an embodiment of the present invention;

FIG. 4 shows a block diagram that illustrates a system according to illustrative embodiments;

FIG. 5 shows a logical flow diagram according to illustrative embodiments;

FIG. 6 shows a graphically changed digital twin according to illustrative embodiments; and

FIG. 7 depicts a method flow diagram for enhancing a workplace environment using digital twin-based simulation according to an embodiment of the present invention.

The drawings are not necessarily to scale. The drawings are merely schematic representations, not intended to portray specific parameters of the invention. The drawings are intended to depict only typical embodiments of the invention, and therefore should not be considered as limiting the scope of the invention. In the drawings, like numbering represents like elements.

### DETAILED DESCRIPTION

Illustrative embodiments will now be described more fully herein with reference to the accompanying drawings, in which exemplary embodiments are shown. This disclosure may, however, be embodied in many different forms and should not be construed as limited to the embodiments set forth herein. Rather, these illustrative embodiments are provided so that this disclosure will be thorough and complete and will fully convey the scope of this disclosure to those skilled in the art. In the description, details of well-known features and techniques may be omitted to avoid unnecessarily obscuring the presented embodiments.

The terminology used herein is for the purpose of describing particular embodiments only and is not intended to be limiting of this disclosure. As used herein, the singular forms “a”, “an”, and “the” are intended to include the plural forms as well, unless the context clearly indicates otherwise. Furthermore, the use of the terms “a”, “an”, etc., do not denote a limitation of quantity, but rather denote the presence of at least one of the referenced items. It will be further understood that the terms “comprises” and/or “comprising”, or “includes” and/or “including”, when used in this specification, specify the presence of stated features, regions, integers, steps, operations, elements, and/or components, but do not preclude the presence or addition of one or more other features, regions, integers, steps, operations, elements, components, and/or groups thereof. Furthermore, the term “developer” refers to any person who writes computer software. The term can refer to a specialist in one area of computer programming or to a generalist who writes code for many kinds of software.

As indicated above, embodiments of the invention present invention provide an approach for enhancing a workplace environment. Environmental information about the workplace environment is obtained from a plurality of internet of things (IoT) device sensors. Based on the environmental information, a digital twin of the workplace environment is created. The digital twin is evaluated over time using a cognitive system to identify a deficiency in the workplace environment using continuous learned data. Based on the evaluation, an area of an avatar-based version of the digital twin that contains a graphical change in the digital twin containing a suggested improvement that addresses the deficiency in the workplace environment is highlighted.

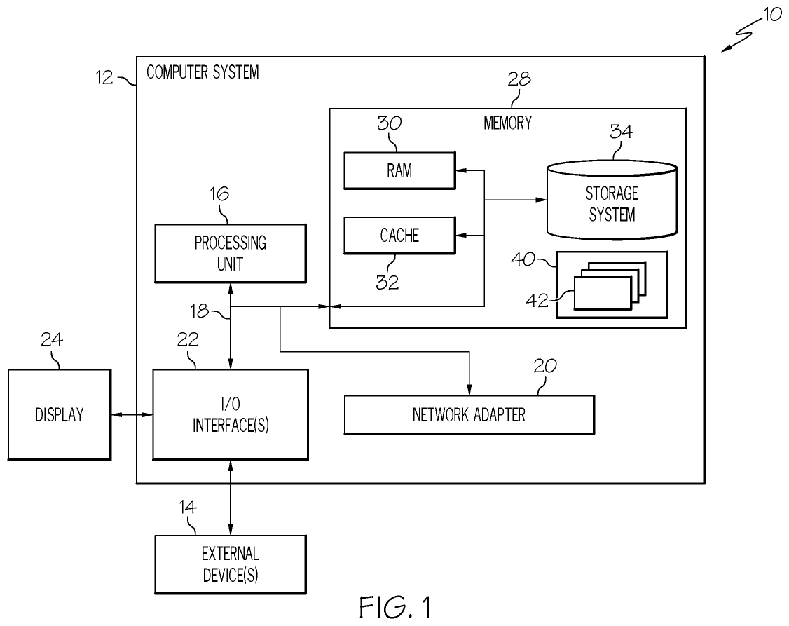

Referring now to FIG. 1, a computerized implementation 10 of an embodiment for enhancing a workplace environment using digital twin-based simulation will be shown and described. Computerized implementation 10 is only one example of a suitable implementation and is not intended to suggest any limitation as to the scope of use or functionality of embodiments of the invention described herein. Regardless, computerized implementation 10 is capable of being implemented and/or performing any of the functionalities set forth hereinabove.

In computerized implementation 10, there is a computer system/server 12, which is operational with numerous other (e.g., special purpose) computing system environments or configurations. Examples of well-known computing systems, environments, and/or configurations that may be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

This is intended to demonstrate, among other things, that the present invention could be implemented within a network environment (e.g., the Internet, a wide area network (WAN), a local area network (LAN), a virtual private network (VPN), etc.), a cloud computing environment, a cellular network, or on a stand-alone computer system. Communication throughout the network can occur via any combination of various types of communication links. For example, the communication links can comprise addressable connections that may utilize any combination of wired and/or wireless transmission methods. Where communications occur via the Internet, connectivity could be provided by conventional TCP/IP sockets-based protocol, and an Internet service provider could be used to establish connectivity to the Internet. Still yet, computer system/server 12 is intended to demonstrate that some or all of the components of implementation 10 could be deployed, managed, serviced, etc., by a service provider who offers to implement, deploy, and/or perform the functions of the present invention for others.

Computer system/server 12 is intended to represent any type of computer system that may be implemented in deploying/realizing the teachings recited herein. Computer system/server 12 may be described in the general context of computer system/server executable instructions, such as program modules, being executed by a computer system. Generally, program modules may include routines, programs, objects, components, logic, data structures, and so on, that perform particular tasks or implement particular abstract data types. In this particular example, computer system/server 12 represents an illustrative system for replicating data records between a source database system and a target database system. It should be understood that any other computers implemented under the present invention may have different components/software, but can perform similar functions.

Computer system/server 12 in computerized implementation 10 is shown in the form of a computing device. The components of computer system/server 12 may include, but are not limited to, one or more processors or processing units 16, a system memory 28, and a bus 18 that couples various system components including system memory 28 to processing unit 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Processing unit 16 refers, generally, to any apparatus that performs logic operations, computational tasks, control functions, etc. A processor may include one or more subsystems, components, and/or other processors. A processor will typically include various logic components that operate using a clock signal to latch data, advance logic states, synchronize computations and logic operations, and/or provide other timing functions. During operation, processing unit 16 collects and routes signals representing inputs and outputs between external devices 14 and input devices (not shown). The signals can be transmitted over a LAN and/or a WAN (e.g., T1, T3, 56 kb, X.25), broadband connections (ISDN, Frame Relay, ATM), wireless links (802.11, Bluetooth, etc.), and so on. In some embodiments, the signals may be encrypted using, for example, trusted key-pair encryption. Different systems may transmit information using different communication pathways, such as Ethernet or wireless networks, direct serial or parallel connections, USB, Firewire®, Bluetooth®, or other proprietary interfaces. (Firewire is a registered trademark of Apple Computer, Inc. Bluetooth is a registered trademark of Bluetooth Special Interest Group (SIG)).

In general, processing unit 16 executes computer program code, such as program code for replicating data records between a source database system and a target database system, which is stored in memory 28, storage system 34, and/or program/utility 40. While executing computer program code, processing unit 16 can read and/or write data to/from memory 28, storage system 34, and program/utility 40.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media, (e.g., VCRs, DVRs, RAID arrays, USB hard drives, optical disk recorders, flash storage devices, and/or any other data processing and storage elements for storing and/or processing data). By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and/or an optical disk drive for reading from or writing to a removable, non-volatile optical disk such as a CD-ROM, DVD-ROM, or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program code embodied on a computer readable medium may be transmitted using any appropriate medium including, but not limited to, wireless, wireline, optical fiber cable, radio-frequency (RF), etc., or any suitable combination of the foregoing.

Program/utility 40, having a set (at least one) of program modules 42, may be stored in memory 28 by way of example, and not limitation. Memory 28 may also have an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a consumer to interact with computer system/server 12; and/or any devices (e.g., network card, modem, etc.) that enable computer system/server 12 to communicate with one or more other computing devices. Such communication can occur via I/O interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples include, but are not limited to: microcode, device drivers, redundant processing units, external disk drive arrays, RAID systems, tape drives, and data archival storage systems, etc.

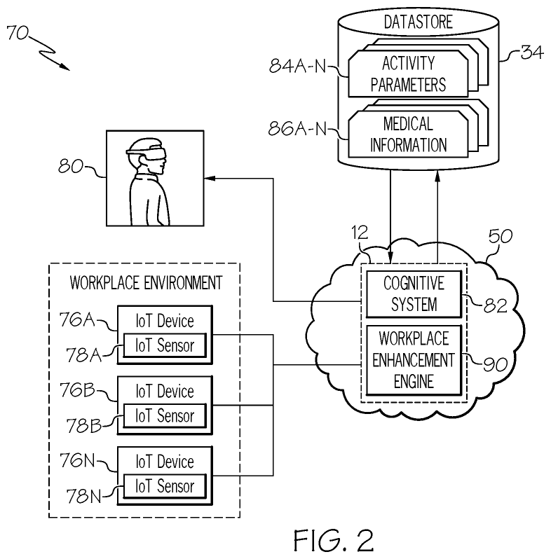

Referring now to FIG. 2, a system diagram describing the functionality discussed herein according to an embodiment of the present invention is shown. It is understood that the teachings recited herein may be practiced within any type of networked computing environment 70 (e.g., a cloud computing environment 50). A stand-alone computer system/server 12 is shown in FIG. 2 for illustrative purposes only. In the event the teachings recited herein are practiced in a networked computing environment, each client need not have a workplace enhancement engine (hereinafter “system 90”). Rather, all or part of system 90 could be loaded on a server or server-capable device that communicates (e.g., wirelessly) with the clients to provide for enhancing a workplace environment using digital twin-based simulation.

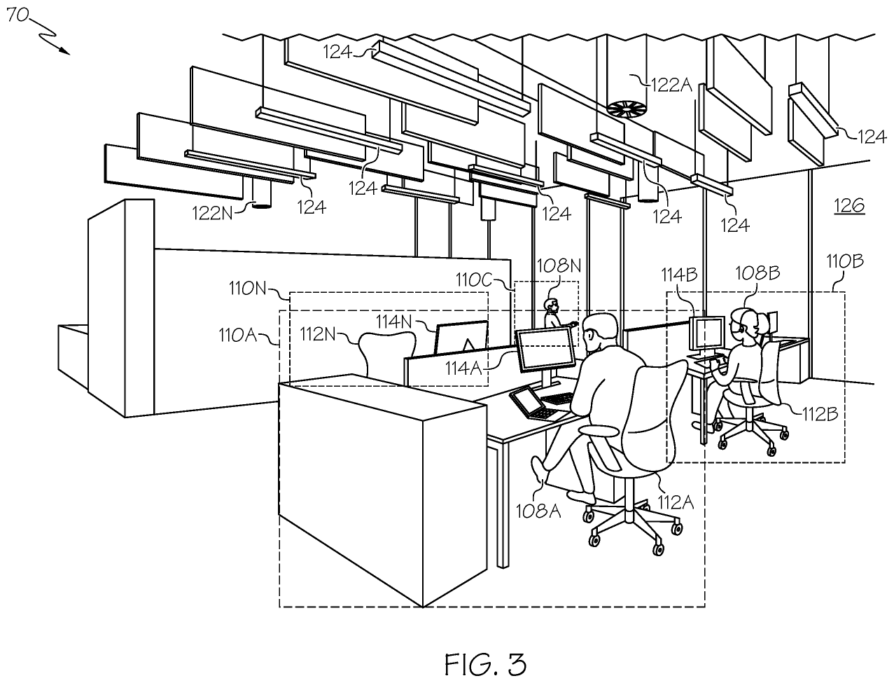

Referring now to FIG. 3, an example workplace environment 70 according to an embodiment of the present invention is shown. In the illustrated example, workplace environment is depicted as an open office environment. However, it should be understood that workplace environment could include any environment in which a worker 108N (hereinafter generic singular 108N, generic plural 108A-N) performs a work task. To this extent, workplace environment can include a traditional office environment, an open office environment, a production environment, an industrial environment, a workshop environment, a warehouse environment, a construction environment, and/or any other indoor or outdoor environment in which a worker performing a work task may be located.

As illustrated, workplace environment 70 includes a number of individual work areas 110A-N (hereinafter generic singular 110N, generic plural 110A-N), with a worker 108N being assigned to each individual work area 110N. It should be understood that the boundaries of a particular individual work area need not be exclusive of all other individual work areas 110A-N, but, rather, the boundaries of two or more work areas 110A-N may overlap, such that the workers 108A-N assigned to the overlapping work areas 110A-N are required to share all or a portion of their individual work areas 110A-N. Moreover, in a shared space workplace solution, a plurality of different workers 108A-N may be assigned to the same individual work area over different periods of time. In any case, each individual work area 110N can include one or more internal features. Internal features can be thought of as objects, structures, and/or the like that are located within the individual work area 110N that can impact the work task being performed by worker 108N. As shown, each individual work area 110N has internal features that can include a seating apparatus 112N (hereinafter generic singular 112N, generic plural 112A-N) and a computing device 114N (hereinafter generic singular 114N, generic plural 114A-N), among others.

In addition, workplace environment 70 can contain a number of external features. External features can be thought of as objects, structures, and/or the like that are located outside of the individual work area 110N that can impact the work task being performed by worker 108N. As illustrated, work environment 100 has external features that include a number of temperature regulation sources 122A-N and a number of man-made light sources 124 and natural light sources 126.

The inventors of the invention described herein have discovered a number of deficiencies in the way in which workplace environments are designed and/or maintained. Workers in different working environments may be required to perform significantly different types of activities, with some of these activities being manual, some automatic, and some semi-automatic. A workplace that provides the worker with a better working experience and that has better ergonomics makes the worker more productive and produces increased worker satisfaction. In addition, improper workplace design is known to cause a number of workplace ailments, which increase worker dissatisfaction and reduce worker productivity. Current solutions for addressing these issues are limited by the fact that each individual is different. Because of this, one-size-fits-all solutions may be ineffective. Rather, a solution that provides a benefit to one worker may provide no benefit or may instead negatively impact another worker. Additionally, conditions that are detrimental to worker health, satisfaction, and/or productivity may not be instantly ascertainable, but may instead only be realized over time, if at all.

The invention described herein utilizes a digital twin to analyze a workplace, identify a deficiency in the workplace, and to alter the digital twin to include a virtual fixing of the deficiency that can then be applied to the real-world workplace. A digital twin is a digital replica of a product, process, or service. This living model creates a thread between the physical and digital world. IoT-connected objects are replicated digitally, enabling simulations, testing, modeling and monitoring based on data collected by IoT sensors. Like everything in the realm of IoT, data is the primary driver, and most invaluable output, of digital twins. The sharing and analysis of digital twin data can empower a user to make decisions which directly impact her concentration and/or performance.

One advantage of the solution provided by the present invention is that it allows the user to identify and remove/reduce any potential deficiencies in the workplace environment, which can have a negative impact on a user's satisfaction and/or performance. By identifying these deficiencies and by providing a virtual space that includes an improvement that resolves these deficiencies, the current invention can provide the user with a tool that allows the user to evaluate the workplace improvement in the context of the workplace environment. The resulting reduction of workplace deficiencies creates an environment in which the worker is more satisfied and more productive.

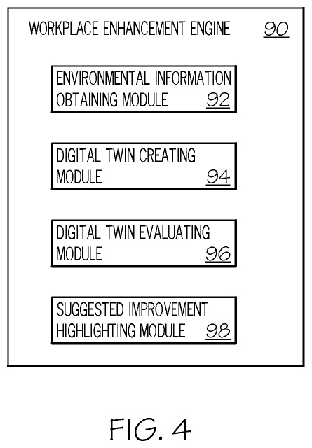

Referring now to FIG. 4, a block diagram that illustrates system 90 is depicted according to illustrative embodiments. It should be understood that system 90 can be implemented as program/utility 40 on computer system 12 of FIG. 1 and can enable the functions recited herein. Along these lines, system 90 may perform multiple functions. Specifically, among other functions, system 90 can enhance a workplace environment using digital twin-based simulation in a networked computing environment. To accomplish this, system 90 can include a set of components (e.g., program modules 42 of FIG. 1) for carrying out embodiments of the present invention. These components can include, but are not limited to, environmental information obtaining module 92, digital twin creating module 94, digital twin evaluating module 96, and suggested improvement highlighting module 98.

Referring now to FIG. 2 in conjunction with FIGS. 3 and 4, environmental information obtaining module 92, as executed by computer system/server 12, is configured to obtain environmental information about workplace environment 70 from a plurality of internet of things (IoT) device 76A-N sensors 78A-N. Broadly speaking, sensors 78A-N are devices that detect and respond to changes in an environment. Inputs can come from a variety of sources such as light, temperature, motion, and pressure. A sensor 78A-N measures a physical quantity and converts it into a signal. Sensors 78A-N translate measurements from the real world into data for the digital domain. There are a vast array of parameters that can be measured, such as location, displacement, movement, sound frequency, temperature, pressure, humidity, electrical voltage level, camera images, color, chemical composition, etc.

Though this disclosure pertains to the collection of personal data (e.g., workplace data), it is noted that in embodiments, users opt-in to the system (e.g., workplace enhancement engine 90). In doing so, they are informed of what data is collected and how it will be used, that any collected personal data may be encrypted while being used, that users can opt-out at any time, and that if they opt-out, any personal data of the user is deleted.

In order to enable sensors for obtaining the environmental information, once a user 80, who wants to utilize the functionality described herein has opted in, environmental information obtaining module 92 can enroll any number of IoT devices 76A-N within workplace environment 72. This enrolling can allow for automated discovery and identification of each IoT device 76A-N, rather than requiring manual input of device identifiers. The process may be similar to Bluetooth or network discovery tools on computers and mobile devices, or the like. Once the available devices within workplace environment 72 are identified, user 80 can choose to add the devices into a central registry. This updated list of devices reflects the enrolled set of devices that can be used for digital-twin simulation. Alternatively, or in addition, manual input of IoT device identifiers can be used.

IoT devices 76A-N within workplace environment 72 can by classified into a number of different types. Consumer-related IoT devices can include smart TVs, smart speakers, toys, wearables, and smart appliances. Smart meters, commercial security systems and smart city technologies, such as those used to monitor traffic and weather conditions, are examples of industrial and enterprise IoT devices. Other technologies, including smart air conditioning, smart thermostats, smart lighting, and smart security, can span home, enterprise, and industrial uses. As discussed, IoT devices are nonstandard computing devices that connect wirelessly to a network and have the ability to transmit data. IoT typically involves extending Internet connectivity beyond standard devices, such as desktops, laptops, smartphones, and tablets, to any range of traditionally non-Internet-enabled physical devices and everyday objects. Embedded with technology, these devices can communicate and interact over the Internet. Connected devices are part of an ecosystem in which every device can talk to other related devices in an environment to automate home or industry tasks. They can communicate usable sensor data to users, businesses, and other intended parties.

In any case, environmental information obtaining module 92 can capture device data from each enrolled IoT device 76A-N. Device data can include, but is not limited to, device type, functionalities and capabilities of each device, any workflows among the devices, location and/or mobility of each device, usage behavior of each device, and/or the like. While some IoT devices, such as a thermostat or cleaning robot, can impact any person within a smart environment, mobile IoT devices (e.g., smart watch, belt, or phone) are typically associated with the person carrying the device. Therefore, for any mobile devices enrolled in the system, device enrolling module 52 can also capture an identity of the opted-in person (e.g., person's name, unique identifier, etc.) using the device which can prove useful when analyzing data collected by the device. The association process can be lightweight and as simple as selecting the person's devices from a list of recognized devices having been previously enrolled. Other devices, particularly non-mobile devices such as a cleaning robot, can be associated with all persons within the smart workplace environment 72.

In any event, each of IoT devices 76A-N that have been deployed in workplace environment 72 has at least one communications component controlled by the processor of the IoT device 76A-N. Communications component includes a hardware communicator and a software agent that includes standard protocols used in the IoT environment. To this extent, communications component is designed to be able to process communications from IoT devices 86A-N, determine the protocols of the processed communications, and interpret the communications in order to facilitate interoperability among IoT devices 86A-N. In order to accomplish this, a single communications component can be designed to process a single type of communication, multiple types of communications, or all types of communications technologies. Communications components in multiple IoT devices 86A-N can collaborate to determine the type of communication used to share the collected environmental information.

Referring back to FIG. 2, sensor data collected from certain IoT devices 76A-N (e.g., smart phone, smart watch, etc.) can be associated with a specific opted-in person within the smart environment based on the device enrollment/association process discussed earlier. Based on which mobile smart devices (e.g., smart watch, smart belt, etc.) the person is using, information such as location, physical movement, fall detection, fatigue, appetite, sleeping patterns, vital signs (heart rate, blood pressure, oxygen levels, etc.), and/or the like, can be collected.

Digital twin creating module 94, as executed by computer system/server 12, is configured to create a digital twin of the workplace environment based on the environmental information obtained by environmental information obtaining module 92 from sensors 78A-N of IoT devices 76A-N. As stated, in its basic form, a digital twin is the digital representation of physical or non-physical processes, systems, or objects. The real-time digital representation a digital twin provides serves as a world of its own. Within this digital world, all types of simulation can be run. Simulations can help a user understand what may happen in the real world by enabling accurate prediction and what-if analysis. Digital twin simulations can be viewed using a display (e.g., mobile device or computer screen, etc.), virtual reality headset, or the like. By understanding real world device behavior using simulations, the user can then use the digital twin, instead of an actual physical device, to make adjustments and visualize any changes in the digital twin in response to the adjustments.

To accomplish this, digital twin creating module 94 can identify from the environmental information data worker-specific information from the IoT devices that obtain information specific to the worker 108N (e.g., wearables, smart medical equipment, smart watch, smart phone, external camera, microphone, pressure sensor, accelerometer and/or the like). This information can include heart rate data, body temperature data, respiration data, eye tracking data, movement data, sound data, obtained body posture data, weight data, pressure data, and/or the like. Digital twin creating module 94 can utilize this worker-specific information to generate and update an enhanced avatar of worker 108N that is a digital twin of worker 108N in real time. This enhanced avatar can be augmented with the worker-specific information, such as real-time biometric data of worker 108N.

In addition, digital twin creating module 94 can identify from the environmental information data general environment data from the IoT devices that can obtain general information about workplace environment (e.g., smart thermostats, smart lighting, temperature sensors, camera, microphone, pressure sensor, and/or the like). This information can include object location, worker location, worker movement, temperature, light levels, light origination, air flow, air quality, noise, and/or the like. Digital twin creating module 94 can utilize this general information to generate the digital twin of the workplace environment 70 in which worker 108N is located including, but not limited to every machine, seating place, walking place, activity area, type of activity currently being performed, and/or any other internal features of individual work area 110N associated with worker 108N and/or external features of workplace environment 70 as a whole. The digital twin can also be augmented to display information associated with the general information including, but not limited to, temperature, luminosity, air quality, noise measurements, air flow, and/or the like.

Digital twin creating module 94 can also identify from the environmental information any interactions between worker 108N and the workplace environment 70. This identifying can include how worker 108N interacts with any machine, object, etc., within workplace environment 70, what type of movement is required in conjunction with the interaction, etc.

In any case, based on this worker-specific information and general information, digital twin creating module 94 can create and continuously update a virtual twin simulation that simulates the worker in real time in a real time virtual representation of workplace environment 70. In conjunction with the execution of the digital twin of worker 108N, digital twin creating module 94 can identify activity parameters 84A-N for each specific worker 108N in workplace environment 70. These activity parameters 84A-N can include worker 108N working pattern, types of movement used, amount of physical force required to be expended and/or the like. These activity parameters 84A-N can be used to create a knowledge corpus (e.g., in datastore 34) that is specific to each worker 108N. Each worker-based knowledge corpus can detail activities being performed by the specific worker 108N based on the activity parameters 84A-N. These activity parameters 84A-N can be combined to create a workflow sequence for the specific worker 108N. Moreover, as new activity parameters 84A-N are added to the knowledge corpus, digital twin creating module 94 can utilize this new information to continuously update the digital twin that is specific to the worker 108N.

In addition, digital twin creating module 94 can create other digital twins that are specific to other workers 108A-N. To this extent, a separate digital twin that is specifically focused on each particular worker 108N in workplace environment 70 can be generated for every worker in workplace environment. These individual worker-specific digital twins can be combined to form a workplace-wide digital twin that combines the perspectives and focus points of the individual digital twins and that can be monitored by an overseeing user 80 (e.g., a supervisor).

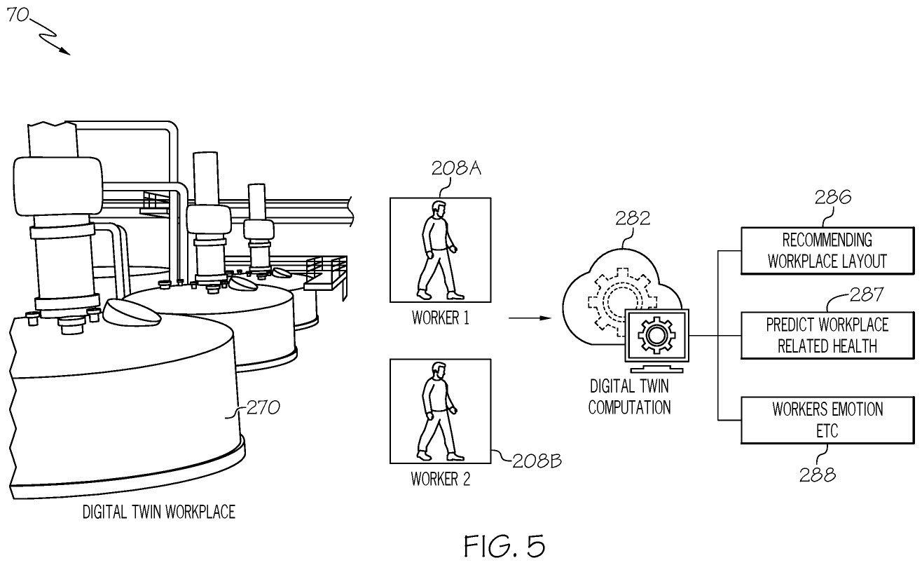

Referring now to FIG. 5, a logical flow diagram 300 is shown according to illustrative embodiments. As shown, a digital twin has been created for each of worker 1 208A and worker 2 208B, and these digital twins have been combined to form digital twin workplace 270.

Referring again to FIG. 4 in conjunction with FIGS. 2, 3 and 5, digital twin evaluating module 96, as executed by computer system/server 12, is configured to evaluate the digital twin 270 over time using a cognitive system 82. Cognitive system 82 includes a cognitive engine, such as IBM's Watson Search or Watson Explorer (IBM is a registered trademark and Watson is a trademark of International Business Machines Corporation) that utilizes machine learning to evaluate the data contained in digital twin, as well as the environmental information used to create based on continuous learned data to identify a deficiency in the workplace environment. Cognitive system 82 is trained via a set of training data to identify the deficiencies in workplace environment 70. Once trained, cognitive system 82 can evaluate the digital twin to identify factors that may be affecting the productivity, harming the heath, or otherwise detracting from the workplace experience of the worker. In embodiments, cognitive system 82 may have access to medical information 86A-N regarding a number of issues that may negatively impact workers. This medical information 86A-N may include medical diagnostic data in the form of continuous learned data, which may be general medical diagnostic data or user-specific medical diagnostic data that the user has consented to have used to improve the user's workplace environment. In response to the identification of such a defect, digital twin evaluating module 96 can notify worker 108N, a supervisor, or any other person who may be designated.

In an embodiment, cognitive system 82 can identify a change or anomalous reading in worker's 108N biometric data collected by biometric sensors that are included in IoT device 76A-N sensors 78A-N. Cognitive system 82 can evaluate this information based on general environmental information collected by environmental sensors in the immediate work environment of worker 108N. Cognitive system 82 can further evaluate the information based on the medical diagnostic data to determine an environmental data reading that is causing the reading of the biometric data.

For example, assume that the biometric sensors associated with worker 1 208A begin to indicate that worker 1 208A is moving more slowly, that worker 1 displays facial expressions and bodily actions that indicate increased agitation, that worker 1 208A in the digital twin has begun to utilize tissues more frequently, and that the oxygen levels in the blood of worker 1 208A have decreased. Cognitive system 82 can use the information in the digital twin based on the environmental sensors to determine that worker 2 208B has recently arrived and that the air in the immediate work environment of worker 208A now includes an increasing amount of aerosol matter. Based on, the medical diagnostic data, cognitive system 82 may make a determination that worker 2 208B may have used a product having a scent to which worker 1 208A is allergic and may identify this as a workplace deficiency.

In another example, the set of environmental sensors may include a microphone that detects the presence of a forceful respiratory discharge (e.g., a cough, sneeze, and/or the like) by worker 1 208A. Further, the set of biometric sensors may include a body temperature sensor that detects that worker 1 208A has an elevated body temperature. Cognitive system 82 may make a determination based on the medical diagnostic data that worker 1 208A have a respiratory illness and may identify this factor as a workplace deficiency.

Suggested improvement highlighting module 98, as executed by computer system/server 12, is configured to highlight an area of an avatar-based version of the digital twin that contains a graphical change in the digital twin. This graphical change within the digital twin contains a suggested improvement that addresses the deficiency in the workplace environment (e.g., in an attempt to resolve it). To accomplish this, suggested improvement highlighting module 98 utilizes a cognitive system 82 based digital twin simulation engine 282 to create one or more alternate digital twin simulations that alter various parameters of the original digital twin. This alteration can include a change in the workflow sequence of the original digital twin that simulates how multi-worker activity synchronization can be performed more effectively given the current workplace setup. This alteration can further include simulated changes in the workplace setup that predict how these changes will affect worker 108N based on how the worker 108N is affected in the digital twin.

Based on these alternate digital twin simulations, suggested improvement highlighting module 98 can recommend alternatives that address the deficiency and improve the workplace environment 70. These alternatives are generated by digital twin simulation engine 282, which identifies an effective workplace design from the alternate digital twin simulations that generates optimal or improved simulation results compared with the original digital twin with respect to the workflow, ergonomics, workplace experience, worker health, etc. Additionally, digital twin simulation engine 282 uses the environmental information to identify the emotional state 288 of workers 108A-N with the current setup based on the original digital twin and simulates the emotional state 288 of workers 108A-N given the new layout in alternate digital twin simulations to determine whether the new layout can bring additional emotional wellbeing and/or excitement, with results impacting whether a new recommended workplace layout 286 is submitted. Moreover, the alternate digital twin simulations can be used by digital twin simulation engine 282 to simulate instances in which elements of the current workplace environment 70 may cause worker 108N to experience workplace related health 287 issues and can recommend an improvement that that provides a proactive treatment therefor.

In any case, the improvement can include a change to the layout of individual work area 110N assigned to worker 108N, a change to the layout of the workplace environment as a whole, or a combination of the two. For example, in the example in which worker 1 208A was having an allergic reaction, the change recommended by suggested improvement highlighting module 98 may include the inclusion of an air purifier in the individual work area 110N of worker 1 208A, a relocation of worker 1 208A or worker 2 208B, etc. In an example in which digital twin simulation engine 282 determines that worker 1 208A is not sitting properly, which could lead to back problems, the change recommended by suggested improvement highlighting module 98 may include the replacement of the chair of worker 1 208A, the raising or lowering of the work surface of worker 1 208A, etc. In another example in which digital twin simulation engine 282 determines that there is a glare on the monitor of worker 1 208A, which could lead to eye strain, the change recommended by suggested improvement highlighting module 98 may include installation of a shade over external light source 126, installation of a glare screen on the monitor of worker 1 208A, the relocation of the monitor of worker 1 208, etc. Moreover, in the example in which worker 1 208A was determined to have a respiratory illness, the change recommended by suggested improvement highlighting module 98 may include a temporary removal of worker 1 208A to a health care area.

Whatever the change recommended by suggested improvement highlighting module 98, suggested improvement highlighting module 98 provides a virtual user interface for user 80 to view the suggested improvement within the workplace environment 70 provided by the virtual twin. In an embodiment, this virtual user interface can include a three-dimensional avatar-based version of the digital twin in which a graphical change containing the suggested improvement is highlighted (e.g., with a glow, an augmented color, and/or the like. This environment can be accessed by user 80 wearing virtual reality (VR) eyewear (e.g., VR goggles, VR glasses, and/or the like) or utilizing any other solutions for accessing a three-dimensional avatar-based environment that currently exists or is later developed. In any case, the virtual user interface provided by suggested improvement highlighting module 98 allows user 80 to see the improvement within the virtual twin and to simulate the change that the improvement will bring to workplace environment 70.

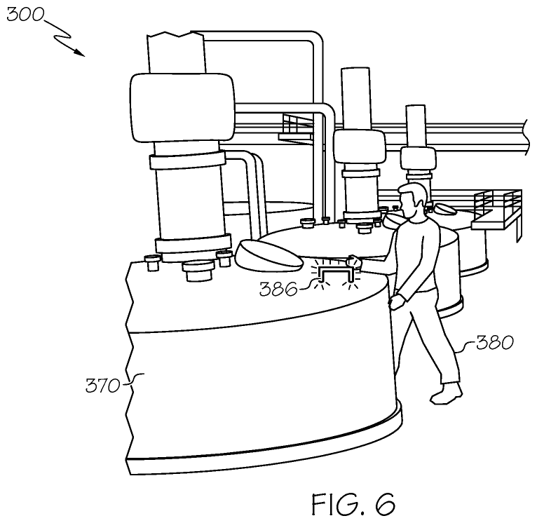

Referring now to FIG. 6, an illustration 300 of a graphically changed digital twin 370 is shown according to illustrative embodiments. Graphically changed digital twin 370 is a digital twin of a workplace environment to which a modification 386 has been made. As illustrated, modification 386 is the addition of a handle, presumably to provide a workplace environment that has been ergonomically enhanced (e.g., optimized) for the worker, whose avatar 380 is shown within graphically changed digital twin. As shown, modification 386 has been highlighted to accentuate the fact that it is an addition.

In any case, referring again to FIGS. 2, 3 and 5, after a change recommended by suggested improvement highlighting module 98 has been implemented, that information is fed back into environmental information obtaining module 92 and incorporated into the digital twin created by digital twin creating module. The results from this new digital twin are compared against the predictions from the alternate digital twin simulation used to make the recommendation to compare the effectiveness of the change to the predicted effectiveness. Using this information, cognitive system 82 and digital twin simulation engine 282 based thereon can utilize machine learning to dynamically incorporate the information into future decision-making processes, resulting in improved future recommendations to the user 80.

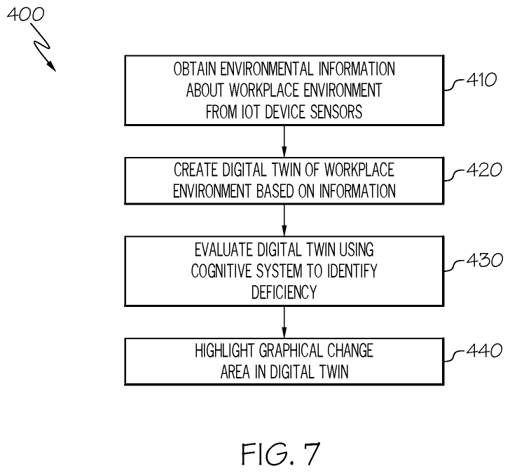

FIG. 7 depicts a method flow diagram 400 for enhancing a workplace environment using digital twin-based simulation according to an embodiment of the present invention. Referring additional to FIGS. 2 and 4, at 410, an environmental information about workplace environment 70 is obtained from a plurality of IoT device 76A-N sensors 78A-N. At 420, a digital twin of workplace environment 70 is created based on the environmental information. At 430, the digital twin is evaluated over time using cognitive system 82 to identify a deficiency in workplace environment 70. At 440, an area of an avatar-based version of the digital twin that contains a graphical change containing a suggested improvement is highlighted.

It will be appreciated that the method process flow diagram of FIG. 7 represents possible implementations of process flows for enhancing a workplace environment using digital twin-based simulation, and that other process flows are possible within the scope of the invention. The method process flow diagrams discussed above illustrate the architecture, functionality, and operation of possible implementations of systems, methods, and computer program products according to various embodiments of the present invention. In this regard, each portion of each flowchart may represent a module, segment, or portion of code, which comprises one or more executable instructions for implementing the specified logical function(s). It will also be noted that each block of each flowchart illustration can be implemented by special purpose hardware-based systems that perform the specified functions or acts.

Further, it can be appreciated that the approaches disclosed herein can be used within a computer system for enhancing a workplace environment using digital twin-based simulation. In this case, as shown in FIG. 1, communication content tool 150 can be provided, and one or more systems for performing the processes described in the invention can be obtained and deployed to computer infrastructure 102 (FIG. 1). To this extent, the deployment can comprise one or more of: (1) installing program code on a computing device, such as a computer system, from a computer-readable storage medium; (2) adding one or more computing devices to the infrastructure; and (3) incorporating and/or modifying one or more existing systems of the infrastructure to enable the infrastructure to perform the process actions of the invention.

The exemplary computer system 12 (FIG. 1) may be described in the general context of computer-executable instructions, such as program modules, being executed by a computer. Generally, program modules include routines, programs, people, components, logic, data structures, and so on, which perform particular tasks or implement particular abstract data types. Exemplary computer system 12 may be practiced in distributed computing environments where tasks are performed by remote processing devices that are linked through a communication network. In a distributed computing environment, program modules may be located in both local and remote computer storage media including memory storage devices.

Some of the functional components described in this specification have been labeled as systems or units in order to more particularly emphasize their implementation independence. For example, a system or unit may be implemented as a hardware circuit comprising custom VLSI circuits or gate arrays, off-the-shelf semiconductors such as logic chips, transistors, or other discrete components. A system or unit may also be implemented in programmable hardware devices such as field programmable gate arrays, programmable array logic, programmable logic devices or the like. A system or unit may also be implemented in software for execution by various types of processors. A system or unit or component of executable code may, for instance, comprise one or more physical or logical blocks of computer instructions, which may, for instance, be organized as an object, procedure, or function. Nevertheless, the executables of an identified system or unit need not be physically located together, but may comprise disparate instructions stored in different locations which, when joined logically together, comprise the system or unit and achieve the stated purpose for the system or unit.

Further, a system or unit of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices and disparate memory devices.

Furthermore, systems/units may also be implemented as a combination of software and one or more hardware devices. For instance, System 90 may be embodied in the combination of a software executable code stored on a memory medium (e.g., memory storage device). In a further example, a system or unit may be the combination of a processor that operates on a set of operational data.

As noted above, some of the embodiments may be embodied in hardware. The hardware may be referenced as a hardware element. In general, a hardware element may refer to any hardware structures arranged to perform certain operations. In one embodiment, for example, the hardware elements may include any analog or digital electrical or electronic elements fabricated on a substrate. The fabrication may be performed using silicon-based integrated circuit (IC) techniques, such as complementary metal oxide semiconductor (CMOS), bipolar, and bipolar CMOS (BiCMOS) techniques, for example. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor devices, chips, microchips, chip sets, and so forth. However, the embodiments are not limited in this context.

Also noted above, some embodiments may be embodied in software. The software may be referenced as a software element. In general, a software element may refer to any software structures arranged to perform certain operations. In one embodiment, for example, the software elements may include program instructions and/or data adapted for execution by a hardware element, such as a processor. Program instructions may include an organized list of commands comprising words, values, or symbols arranged in a predetermined syntax that, when executed, may cause a processor to perform a corresponding set of operations.

The present invention may also be a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

It is apparent that there has been provided with this invention an approach for enhancing a workplace environment using digital twin-based simulation. While the invention has been particularly shown and described in conjunction with a preferred embodiment thereof, it will be appreciated that variations and modifications will occur to those skilled in the art. Therefore, it is to be understood that the appended claims are intended to cover all such modifications and changes that fall within the true spirit of the invention.

What is claimed is:

1. A computer-implemented method for enhancing a workplace environment, comprising the computer-implemented steps of:
obtaining environmental information about the workplace environment from a plurality of internet of things (IoT) device sensors;
creating a digital twin of the workplace environment based on the environmental information;
evaluating the digital twin overtime using a cognitive system to identify a deficiency in the workplace environment using continuous learned data; and
highlighting an area of an avatar-based version of the digital twin that contains a graphical change in the digital twin containing a suggested improvement that addresses the deficiency in the workplace environment.

2. The computer-implemented method of claim 1, the creating further comprising:
identifying activity parameters of a first worker in the workplace environment that includes a working pattern of the first worker, types of movement used by the first worker, and an amount of physical force required to be expended by the first worker;
creating a knowledge corpus specific to the first worker that details activities being performed by the first worker based on the activity parameters; and
continuously updating the digital twin using the knowledge corpus, the digital twin being specific to the first worker.

3. The computer-implemented method of claim 2, further comprising creating a plurality of other digital twins, each of the plurality of other digital twins being specific to a particular worker in the workplace environment such that a separate digital twin is generated for every worker in the workplace environment.

4. The computer-implemented method of claim 3, further comprising:
combining the plurality of digital twins to form a workplace-wide digital twin; and
notifying a supervisor in the workplace environment in response to the deficiency being identified.

5. The computer-implemented method of claim 2,
wherein the plurality of IoT device sensors include a set of environmental sensors in an immediate work environment of the first worker,
wherein the plurality of IoT device sensors further include a set of biometric sensors that collect biometric data from the first worker,
wherein the continuous learned data includes medical diagnostic data; and
wherein the evaluating includes the cognitive system determining an environmental data reading that is causing a reading of the biometric data using the continuous learned data.

6. The computer-implemented method of claim 5,
wherein the set of environmental sensors includes a microphone that detects a presence of a forceful respiratory discharge,
wherein the set of biometric sensors includes a body temperature sensor that detects an elevated body temperature,
wherein the evaluating includes a determination by the cognitive system that the first worker has a respiratory illness, and
wherein the graphical change in the digital twin indicated by the highlighted area indicates a removal of the first worker to a health care area.

7. The computer-implemented method of claim 1, further comprising providing a three-dimensional interface to the digital twin that a user accesses using virtual reality eyewear.

8. A system for enhancing a workplace environment, comprising:
a memory medium comprising program instructions;
a bus coupled to the memory medium; and
a processor, for executing the program instructions, coupled to the memory medium that when executing the program instructions causes the system to:
obtain environmental information about the workplace environment from a plurality of internet of things (IoT) device sensors;
create a digital twin of the workplace environment based on the environmental information;
evaluate the digital twin over time using a cognitive system to identify a deficiency in the workplace environment using continuous learned data; and
highlight an area of an avatar-based version of the digital twin that contains a graphical change in the digital twin containing a suggested improvement that addresses the deficiency in the workplace environment.

9. The system of claim 8, the program instructions further causing the system to:
identify activity parameters of a first worker in the workplace environment that includes a working pattern of the first worker, types of movement used by the first worker, and an amount of physical force required to be expended by the first worker;
create a knowledge corpus specific to the first worker that details activities being performed by the first worker based on the activity parameters; and
continuously update the digital twin using the knowledge corpus, the digital twin being specific to the first worker.

10. The system of claim 8, the program instructions further causing the system to create a plurality of other digital twins, each of the plurality of other digital twins being specific to a particular worker in the workplace environment such that a separate digital twin is generated for every worker in the workplace environment.

11. The system of claim 10, the program instructions further causing the system to:
combine the plurality of digital twins to form a workplace-wide digital twin; and
notify a supervisor in the workplace environment in response to the deficiency being identified.

12. The system of claim 9,
wherein the plurality of IoT device sensors include a set of environmental sensors in an immediate work environment of the first worker,
wherein the plurality of IoT device sensors further include a set of biometric sensors that collect biometric data from the first worker,
wherein the continuous learned data includes medical diagnostic data; and
wherein the evaluating includes the cognitive system determining an environmental data reading that is causing a reading of the biometric data using the continuous learned data.

13. The system of claim 12,
wherein the set of environmental sensors includes a microphone that detects a presence of a forceful respiratory discharge,
wherein the set of biometric sensors includes a body temperature sensor that detects an elevated body temperature,
wherein the evaluating includes a determination by the cognitive system that the first worker has a respiratory illness, and
wherein the graphical change in the digital twin indicated by the highlighted area indicates a removal of the first worker to a health care area.

14. The system of claim 8, the program instructions further causing the system to provide a three-dimensional interface to the digital twin that a user accesses using virtual reality eyewear.

15. A computer program product for enhancing a workplace environment, the computer program product comprising a computer readable storage device, and program instructions stored on the computer readable storage device, to:
obtain environmental information about the workplace environment from a plurality of internet of things (IoT) device sensors;
create a digital twin of the workplace environment based on the environmental information;
evaluate the digital twin over time using a cognitive system to identify a deficiency in the workplace environment using continuous learned data; and
highlight an area of an avatar-based version of the digital twin that contains a graphical change in the digital twin containing a suggested improvement that addresses the deficiency in the workplace environment.

16. The computer program product of claim 15, the program instructions stored on the computer readable storage device further to:
identify activity parameters of a first worker in the workplace environment that includes a working pattern of the first worker, types of movement used by the first worker, and an amount of physical force required to be expended by the first worker;
create a knowledge corpus specific to the first worker that details activities being performed by the first worker based on the activity parameters; and
continuously update the digital twin using the knowledge corpus, the digital twin being specific to the first worker.

17. The computer-implemented method of claim 16, the program instructions stored on the computer readable storage device further to create a plurality of other digital twins, each of the plurality of other digital twins being specific to a particular worker in the workplace environment such that a separate digital twin is generated for every worker in the workplace environment.

18. The computer program product of claim 17, the program instructions stored on the computer readable storage device further to:
combining the plurality of digital twins to form a workplace-wide digital twin; and
notifying a supervisor in the workplace environment in response to the deficiency being identified.

19. The computer program product of claim 16,
wherein the plurality of IoT device sensors include a set of environmental sensors in an immediate work environment of the first worker,
wherein the plurality of IoT device sensors further include a set of biometric sensors that collect biometric data from the first worker,
wherein the continuous learned data includes medical diagnostic data; and
wherein the evaluating includes the cognitive system determining an environmental data reading that is causing a reading of the biometric data using the continuous learned data.

20. The computer program product of claim 19,
wherein the set of environmental sensors includes a microphone that detects a presence of a forceful respiratory discharge,
wherein the set of biometric sensors includes a body temperature sensor that detects an elevated body temperature,
wherein the evaluating includes a determination by the cognitive system that the first worker has a respiratory illness, and
wherein the graphical change in the digital twin indicated by the highlighted area indicates a removal of the first worker to a health care area.
